(19) United States
(12) Patent Application Publication
(10) Pub. No.: US 2002/0196895 A1
(43) Pub. Date: Dec. 26, 2002

(54) X-RAY EXAMINATION UNIT FOR TOMOSYNTHESIS

(76) Inventor: JOSEF PLOTZ, MUNICH (DE)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 09/463,216

(22) PCT Filed: Jul. 21, 1998
(86) PCT No.: PCT/EP98/04538

(30) Foreign Application Priority Data
Jul. 24, 1997 (DE) 197 31 927.0

(51) Int. Cl. G21K 1/12; H05G 1/60; A61B 6/00; G01N 23/00

(52) U.S. Cl. 378/21

(57) ABSTRACT

Disclosed is a method and a device enabling x-rax pictures for tomosynthesis to be produced by means of an x-ray examination unit. The method consists in generating radiated pulses from various directions during the radiation detection of an object (3), selecting during the radiation break such signals which can be deflected by a radiation receiver intended for a solid body (2); supplying such signals to a computing and control device (4) and calculating at least one positron emission tomography of at least one predetermined layer.

The invention relates to a method and a device to produce X-ray images for tomosynthesis by means of an X-ray examination unit.

An examination object is thereby penetrated by radiation from various projection directions and the X-ray shadow is imaged on a radiation receiver. Tomograms or three-dimensional images may be calculated by traditional computing methods, particularly in radiation receivers for producing electric signals dependent on incident X-ray shadows. The number of necessary exposures and the solid angle of irradiation are set dependent on the desired depth resolution of the slice (layer) thickness of an image.

A radiation emitter and a radiation receiver are coupled and adjusted and disposed opposite to one another in conventional tomograms (tomography). Objects that lie in the focal plane are sharply imaged since they are projected onto the same location of the radiation receiver during opposed adjustment. Objects that lie outside the focal plane are imaged in blurred fashion since they are projected onto different locations of the radiation receiver during opposed adjustment. The object in the focal plane is imaged onto the radiation receiver by several individual projections at various projection angles α to produce an interpretable exposure. A tomographic image of the object in the focal plane is produced by direct superimposition of the radiation images acquired by the individual projections. A tomographic image of an object that is arranged in a plane parallel to the focal plane may be produced by shifting the radiation images acquired by the individual projections by distance ΔS relative to each other before superimposition. The size and direction of the shift ΔS depends on the position of the radiation emitter and on the location of the plane to be reconstructed.

The shift ΔS for the so-called linear tomography, whereby the radiation emitter is adjusted in one dimension, is determined by the equation:

$$Δ S = \frac{x \cdot h}{x - y - h} \cdot tan α$$

Wherein:

X=distance of the focus of the radiation emitter from the radiation receiver.

H=distance of the focal plane in which an object is to be reconstructed.

Y=distance of the focal plane from the plane of the radiation receiver.

α=projection angle, which means the angle that a reference ray of the ray beam assumes relative to a reference axis, whereby the reference axis is aligned perpendicular to the focal plane.

Literature. Bildgebende Systeme für die medizinische Diagnostik, “Tomosythese”, publisher: Erich Krestel, Verlag Siemens, Berlin/Munich, 2^{nd} Edition, 1988, page 380 and 381.

When the radiation receiver converts the received X-ray shadow of the object into electrical signals, then digital tomosynthesis enables reconstruction of tomography images in a number of planes from the signals of the individual projections of the object that were produced with different projection angles α. Known digital image generating and processing systems can be used in digital tomosynthesis for producing a visible image from the signal of the radiation receiver.

WO 93/22 893 A1 discloses a method whereby it is possible to reconstruct an exposure of an object without knowing the projection angles α and the geometrical configuration of radiation emitter, radiation receiver and focal plane. According to this method, a reference of radiation-absorbent material, having a known size and distance from the radiation receiver, is provided in the region of the radiation receiver and said reference is projected onto the radiation receiver in every individual projection. The geometrical configuration and the two-dimensional projection angle α can be identified on the basis of the local imaging of the reference on the radiation receiver for each individual projection. This reconstruction is time-consuming and complex due to the extensive calculations.

The radiation emitter must assume predetermined positions and alignments relative to the examination object for obtaining an image sequence that can be interpreted tomosynthetically. The alignment can be set, for instance, by an operator of the X-ray examination unit or by employing and driving a radiation emitter that has multiple focuses.

U.S. Pat. No. 5,596,454 and WO 93/22893 disclose X-ray diagnostic devices for producing X-ray exposures for tomosynthesis.

EP 0 632 995 discloses a dental X-ray diagnostic device whereby tomosynthetic exposures of objects may also be produced by the use of a panoramic imaging unit having an X-ray emitter and a receiving unit disposed diametrical opposed to said X-ray emitter. Reference is made to EP 0 229 308 A1 in view of the configuration of traditional panoramic X-ray devices and devices for producing exposures of a scull. The production of panoramic exposures is performed whereby, during the radiation detection of the object (jaw) to be examined, received signals are added in a two-dimensional resolution detection device and whereby the adding of signals may be performed already by this sensor (when a CCD sensor is used) and whereby said sensor is operated in the TDI-mode. Through this special type of operation, the function of a moving film is reproduced whereby the charge packets in the CCD-element, which are produced by exposure, are correspondingly clocked further while new charges are added continuously. The clock pulses for TDI-operation are derived from the step-by-step motor pulses necessary for the film cartridge drive. Furthermore, adding of signals at a later signal-processing phase may also be alternatively possible.

X-ray exposures for tomosynthesis may be produced by deflected and thereby gained signals from the CCD-sensor from various irradiation directions. Should the signals be superimposed to the tomosynthetic reconstruction algorithm, instead of adding them according to TDI pulses, then sharp layers (slices) may be produced with a different and a subsequently determined position. The trade-off is, however, an enormously high rate and amount of data.

The object of the present invention is to avoid these disadvantages and to produce several subsequently-determined sharp image layers (slices) with an adjustment technology corresponding to a traditional panoramic X-ray apparatus and with well-manageable data rates and anoints. An additional object of the present invention is to be able to do without the development of new, special CCD radiation detection devices but to be able to employ instead currently available CCD radiation detection devices used in panoramic X-ray apparatuses, for example.

This object is achieved according to the invention by the characteristics shown in patent claims 1 and 7.

The advantages of the invention is that the radiation pulse is produced from various projection directions during radiation detection of an examination object so that signals, which are deflected by the solid-body receiver during radiation detection, are selected during the radiation break, and whereby the selection of the mechanical adjustment of the imaging unit, consisting of the radiation emitter and the radiation receiver, is decoupled. TDI operation is no longer necessary and the signals, which may be deflected thereby, have no longer a “blurring component”, which would be retained if radiation were produced during the detection process and the imaging unit were thereby adjusted. In addition, already available and known CCD radiation converters may be used.

It is of particular advantage, when radiation detection occurs at a step-by-step change in projection direction, when a radiation pulse is produced at a first projection direction, and when the signals of the solid-body radiation receiver are selected during adjustment to the second projection direction and during the radiation break. The amount of data is thereby reduced since no image signals are produced dung the radiation break and during adjustment to the second projection direction.

For reduction of the blurring effect it is an advantage if the speed for adjustment of the imaging unit, which consists of a radiation emitter and a radiation receiver, is less during radiation pulses as it is during the radiation break.

Should the radiation detection of the object occur by sections, then the computation of a tomogram for one section may already be in progress while radiation detection is still being conducted for at least one other section.

Additional advantages and details of the invention are shown in the following description of an embodiment example with reference to the accompanying drawings and corresponding to the sub-claims:

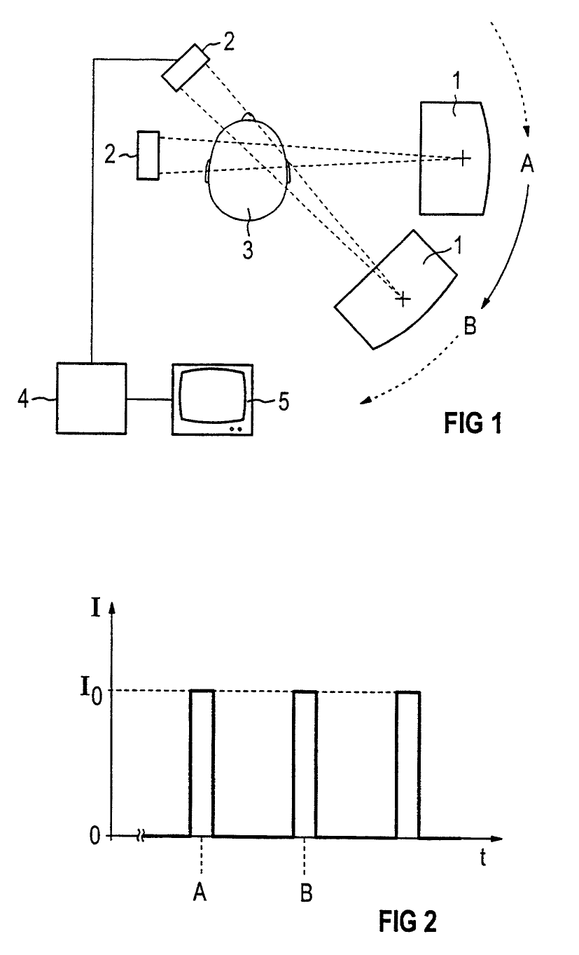

FIG. 1 shows an X-ray examination unit according to the invention in a principal layout.

FIG. 2 shows a diagram of radiation pulses and radiation breaks.

An X-ray examination unit according to the embodiment of the present invention may be used in an application whereby a radiation receiver is used for producing electrical signals, preferably a solid-body radiation detection device, and with which it is possible to scan an object by radiation. Therefore, FIG. 1 shows an X-ray examination unit in only the principle layout, whereby said device is provided with a radiation emitter 1 and a radiation receiver 2, which are part of the imaging unit. The radiation emitter 1 and the radiation receiver 2 are arranged facing each other and are in close relationship with one another. They may be moved around an object 3 by an adjustment device (not further illustrated). Adjustment is performed here by driving a computing and control device 4, which also drives the radiation emitter 1 relative to the production of radiation pulses. The signals of the radiation receiver 2 are supplied to said computing and control device 4, which is designed to compute tomograms and to produce signals so that X-ray exposures for tomosynthesis, in particular, can be displayed on a monitor device 5 connected to the computing and control device 4.

Based on the signals deflected therefore by the radiation receiver 2, individual images in the layers (slices) may be computed so that blurring caused by adjustment movement does no longer occur, or that it is at least reduced,

Furthermore, the pulse duration may be lengthened during radiation and the intensity may be reduced, whereas it would have to be shortened or increased, respectively, if adjustment of the imaging unit is to be continuous.

Driving of the radiation emitter 2 and the adjustment device is performed preferably in such a manner that each of the various projection directions corresponds to a radiation pulse.

Adjustment may therefore be continuous, whereby it must be seen as an advantage when the speed of adjustment, during a radiation pulse generated by the radiation emitter 1, is at least slower than during the radiation break. The adjustment is preferably stopped during the radiation pulse and is continued again during the radiation break.

As previously described, radiation detection is preferably performed by a step-by-step change in projection direction, whereby a radiation pulse is produced at a first projection position A and whereby the signals of the radiation receiver 2 are selected during adjustment to a second projection position B and during the radiation break. It is thereby no longer necessary to operate the radiation receiver 2 in the TDI mode, in which signal integration occurs, since selection occurs during adjustment of the imaging unit and this occurs during the time radiation is switched off. Since signals are produced by a radiation pulse, whereby the imaging unit is preferably stationary, or is at least adjusted at a decreased speed relative to the radiation break, the signals of the radiation receiver 2, and thereby the computable individual images, do no longer include a blurring component.

Should a step motor (pulse motor) be used as an adjustment device, compared to the state-of-the-art, whereby a continuous e.g. pulse-controlled scanning of the object 3 occurs in the TDI mode and whereby in all ten motion clockings only one radiation pulse is produced, by which there is obtained only a six-fold data rate with, for example, a 6 mm wide CCD radiation converter and a pixel size of 0.1 mm together with a six-fold data amount at the same step-motor frequency as in the TDI mode. The peak value of the data rate is reduced, however, by about 2.5-fold if all adjustment phases occur at the same speed, which is made possible by decoupling of the image conversion. The interference and noise contributors originating at the CCD converter and the selection electronics are also low because of the relatively low data rate, so that no significant increased demand in dose is required. These embodiments are of course also valid for any other configuration of the adjustment device.

Radiation pulses of higher intensity but with shorter time periods may be used to decrease radiation detection of the object 3. For the production of signals, radiation pulses may have, for example, a duration of 20 to 30 ms and radiation pulse breaks, which means the selection way have a duration of approximately 50 ms. During scanning of an object 3, which takes for instance a time period of 20 seconds, signals of 300 individual images may be produced, which require about 30 MB of memory space.

The radiation pulses of the radiation emitter 1 may be generated by the corresponding drive of the radiation generator or by regulating an electromechanical radiation shutter. CCD radiation converters with a scintillation layer or aSi, aSe or a CdTe-sensor (detector) may be used as radiation receivers 2.

It may also be of advantage if exposure occurs with very short radiation pulses at continuous adjustment of the imaging unit or a partial TDI pre-integration of signals is intermixed during a normal, fast or slow adjustment speed.

To shorten the time required for the completion of an X-ray exposure for tomosynthesis, it is of an advantage if radiation detection is performed step-by-step, whereby calculation of one tomogram (slice image) for one section is already performed while radiation detection is still being conducted for at least one other section.

1. A method for operating an X-ray examination unit for digital tomosynthesis comprising process steps for
a) generating radiated pulses from various projection directions during radiation detection of an object (3),
b) selecting electric signals deflected by a radiation receiver (2) at radiation break and during radiation detection of radiation shadows of the object (3),
c) supplying said signals to a computing and control device (4),
d) calculating at least one tomogram (slice image) of at least one predetermined layer based on said signals.

2. A method according to claim 1, wherein the radiation pulses are generated by driving a radiation generator or by driving a radiation interruption device (beam chopping device).

3. A method according to claim 1 or 2, wherein radiation detection occurs under a step-by-step change in projection direction,
wherein a radiation pulse is generated at a first projection direction,
and wherein the signals of the radiation receiver (2) are selected during adjustment to a second projection direction and during the radiation break.

4. A method according to claim 3, wherein the adjustment speed of an imaging unit, consisting of a radiation emitter (1) and a radiation receiver (2) of the X-ray examination device, is lower during radiation pulses than during the radiation break.

5. A method according to one of the claims 1 through 4, wherein radiation detection occurs by sections and whereby calculation of one tomogram (slice image) is already performed for one section while radiation detection is still occurring in at least one other section.

6. A method according to one of the claims 1 through 5, wherein the radiation receiver (2) is designed as a CCD, aSi, aSe or as a CdTe-sensor (detector).

7. A device to carry out the process according to one of the claims 1 through 6.
